(19) United States
(12) Patent Application Publication
(10) Pub. No.: US 2007/0073147 A1
(43) Pub. Date: Mar. 29, 2007

(54) Method and apparatus for displaying a measurement associated with an anatomical feature

(75) Inventors: Matthew M. Holladay, Britton, MI (US); Michael Norman Woelmer, Milan, MI (US); John I. Jackson, Menlo Park, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/238,887

(22) Filed: Sep. 28, 2005

(51) Int. Cl. G06K 9/00; A61B 8/00

(52) U.S. Cl. 600/437; 382/128

(57) ABSTRACT

A method and system are provided for displaying a measurement associated with an anatomical feature. The method includes determining, from ultrasound data, a measurement of such feature and assigning a color based on a comparison between the measurement and reference data of such feature. The feature may be, for example, an artery and the measurement the intima media thickness of the artery. In such example, the method and apparatus display vessel wall thickness from ultrasound imaging data and includes determining from the ultrasound imaging data, intima media thickness along the vessel wall and assigning a color based on the intima media thickness determined and a reference intima media thickness.

### TECHNICAL FIELD

This invention relates generally to displaying a measurement associated with an anatomical feature, and more particularly to ultrasound imaging used to display a measurement associated with an anatomical feature such as, for example, vessel wall thickness.

### BACKGROUND

As is known in the art, Intima Media Thickness (IMT) is a measurement of the distance between the lumen-intima (LI) boundary and the media-adventia (MA) boundary of a vessel. Measurement of IMT is an emerging application that is growing in interest and importance to the clinical community. It has been demonstrated as an independent predictor of transient cerebral ischemia, stroke, and coronary events. However, most ultrasound systems that provide an IMT measurement simply provide a measurement, or at best feed that measurement back into risk assessment. So even if an automated measurement is supplied instead of a manual measurement, the resulting numbers must be interpreted in order to perform an assessment of the patient's vascular health.

One technique used to measure IMT is with ultrasound imaging. More particularly, ultrasound imaging data is used to ascertain the LI and MA boundaries (either automatically detected or manually placed), calculate the thickness, and then provide one or more of the following results: the average IMT; the maximum IMT; a graph of the patient's average and/or maximum IMT vs. general population statistics. A risk assessment is made by replacing the patient's chronological age with a vascular age based on how the patient's average IMT relates to the general population statistics. One problem with such method is that all of the results are numeric values that require interpretation. There is no quick visual reference that allows you to easily assess vascular health.

### SUMMARY

In accordance with the present invention, a method and system are provided for displaying a measurement associated with an anatomical feature. The method includes determining, from ultrasound data, a measurement of such feature and assigning a color based on a comparison between the measurement and reference data of such feature.

In one embodiment, the reference data includes population statistics.

In one embodiment, the reference data is a function of prior determined data of the feature.

In accordance with another embodiment, a method is provided for displaying vessel wall thickness from ultrasound imaging data. The method includes determining from the ultrasound imaging data IMT along the vessel wall and assigning a color is based on the IMT measurement and a reference IMT.

In one embodiment the reference IMT is a function of population statistics.

In one embodiment, a single color is assigned to the entire vessel wall based on the average IMT determination and the reference IMT.

In one embodiment, a single color is assigned to the entire vessel wall based on the maximum determined IMT and the reference IMT.

In one embodiment the IMT is determined at each of a plurality of different locations along the vessel wall. The method includes assigning at each one of the different locations along the vessel wall a color based on the IMT determination at each one of the different locations.

In one embodiment the reference IMT is a function of a prior IMT determination.

In one embodiment the reference IMT is a function of a prior IMT determination and the time duration between the determined IMT and the prior IMT determination.

In one embodiment a method is provided for displaying vessel wall thickness from ultrasound imaging data. The method includes generating LI and MA boundaries for the vessel wall; using such ultrasound imaging data; measuring from the generated LI and MA boundaries an IMT along the vessel wall; and assigning a color based on the IMT measurement and the reference IMT.

In one embodiment, a method is provided for displaying vessel wall thickness from ultrasound imaging data taken on a patient. The method includes obtaining demographic information of the patient; obtaining an ultrasound image of the vessel of the patient; determining from the obtained image the IMT of the vessel; comparing the determined IMT with data of IMT for people having substantially similar demographics as the obtained patient demographic information; selecting a color to apply to for displaying the vessel based on the comparison; and generating an image of the patient's vessel with a wall of the vessel image having the selected color.

In one embodiment, an ultrasound system is provided for displaying vessel wall thickness from ultrasound imaging data taken on a patient. The system includes a transducer for obtaining an ultrasound image of the vessel of the patient. The system includes a processor for: determining from the obtained image the IMT of the vessel; comparing the determined IMT with data of IMT for people having substantially similar demographics as the obtained patient demographic information; selecting a color to apply to for displaying a wall of the vessel based on the comparison; and generating an image of the patient's vessel; with the wall of the vessel image having the selected color.

Thus, in accordance with the invention, a method and system are provided for generating LI and MA boundaries for the vessel wall. This may be an automated procedure or a manual procedure. Next the method measures the IMT at each location along the vessel wall. At each location along the vessel wall, a color is assigned based on how the local IMT measurement relates to the general population statistics. For example, the process might assign a green color if the IMT measurement is normal, yellow if it is high, and red if it is very high. Next, the method displays the IMT measurement and then fills in the vessel wall by alpha-blending the color assigned at each location in the image. This will highlight the vessel wall and display a color at each location that clearly identifies if the IMT measurement at that location is normal, high, or very high.

This invention thereby provides a technique for displaying a vessel wall thickness measurement such as IMT to provide a clear visual indication of the patient's vascular health. It does this by shading the area in the image representing the vessel wall thickness using a color map that relates the thickness represented in the image with data from the general population. This gives immediate visual feedback of how the patient's vessel wall thickness relates to the general population. Thus, in addition to providing all of the same measurements as the old methods, this invention allows a quick visual assessment of this particular risk factor. Areas of the vessel wall that have more thickening than is expected in the general population are clearly identified by color on the image, side by side with the actual numeric measurement of IMT.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a sketch of ultrasound apparatus for displaying vessel wall thickness from ultrasound imaging data taken on a patient according to the invention;

FIG. 2 is a block diagram of processing equipment used to the apparatus of FIG. 1 to display vessel wall thickness from ultrasound imaging data taken on a patient according to the invention;

FIG. 3 is a flow diagram of a process used by the apparatus of FIG. 1 for displaying vessel wall thickness from ultrasound imaging data taken on a patient according to the invention;

FIG. 4 is a sketch of an image of a segment of the vessel of the patient obtained with the apparatus of FIG. 1 prior to having a wall thereof displayed with a color selected in accordance with the invention;

FIG. 4A is a sketch of an image of a segment of the vessel of the patient obtained with the apparatus of FIG. 1 after having a wall thereof displayed with a color selected in accordance with the invention; and

FIG. 4B is a sketch of an image of a segment of the vessel of a different patient obtained with the apparatus of FIG. 1 after having a wall thereof displayed with a color selected in accordance with the invention.

Like reference symbols in the various drawings indicate like elements.

### DETAILED DESCRIPTION

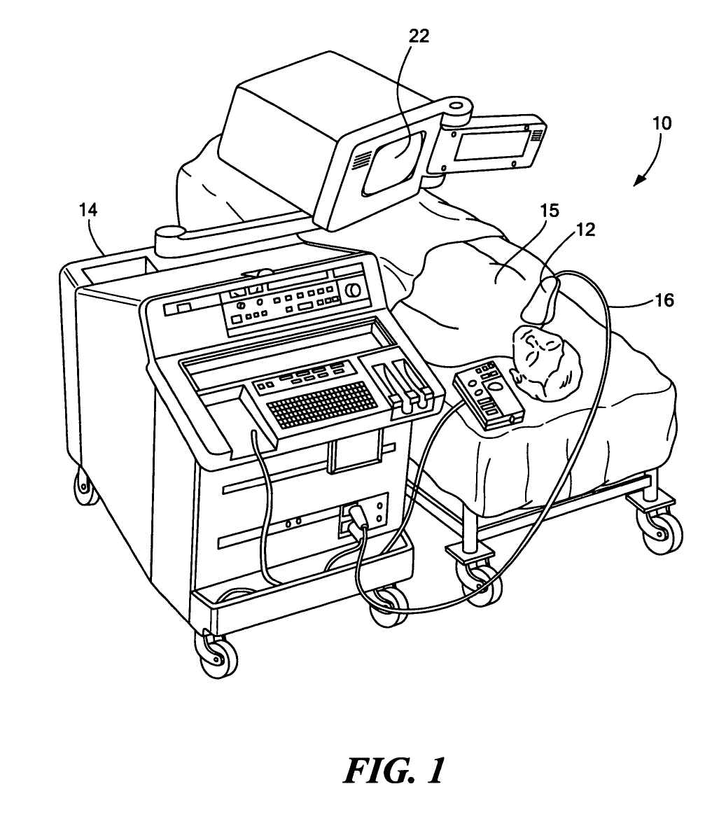

FIG. 1 shows an imaging system 10, here an ultrasound imaging system for medical diagnostics. The system 10 includes a positionable, here handheld, image processing ultrasound device, here a transducer 12 and a multi-use display device, or operator interface or workstation 14. The handheld transducer 12 obtains ultrasound data and formats the ultrasound data for transmission to the workstation 14, here via a cable 16.

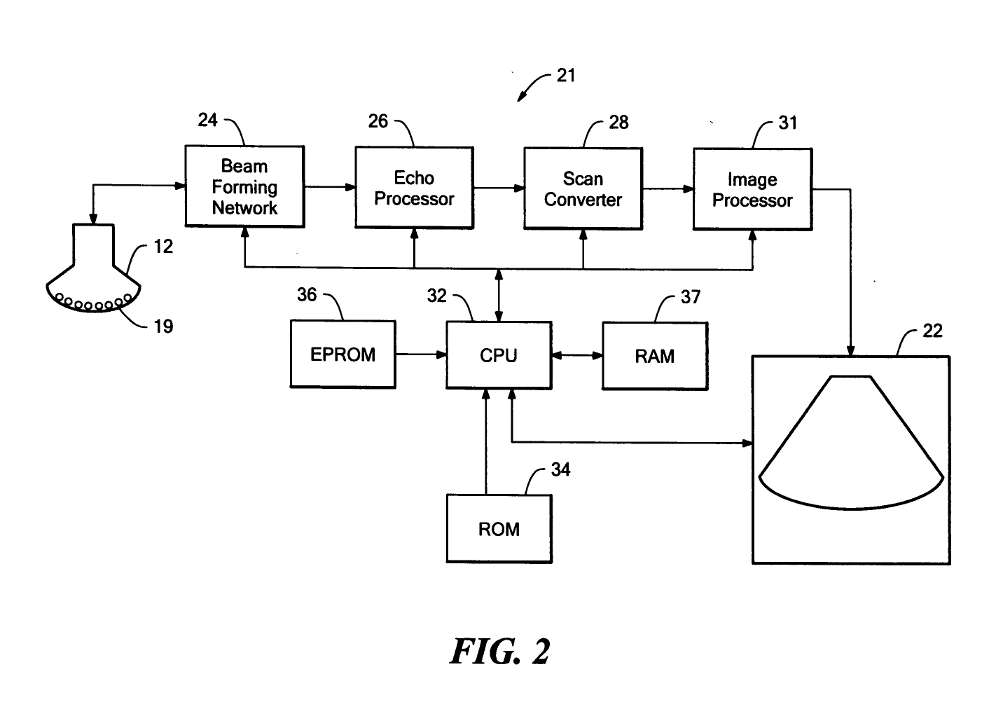

The handheld transducer 12 includes conventional ultrasound circuitry, not shown. Thus, the ultrasound circuitry includes, in the frontal portion thereof an array of ultrasonic elements 19, FIG. 2, which transmit and receive ultrasonic energy for imaging an anatomical feature, here for example, a blood vessel of patient 15, here the carotid artery, not shown, of the patient 15. The elements 19 in the frontal portion fed to a display 22 of the workstation 14 (FIG. 1) serially through: a processor 21, shown in FIG. 2. Thus, the processor 21 includes beamforming network 24 fed by the transducer 12, an echo processor 26, a scan converter 28, and an image processor 31 in a conventional manner. The beamforming network 24, echo processor 26, scan converter 28, image processor 31 and display 22 are controlled by a central processing unit (CPU) 32 coupled to a random access memory RAM 37. The CPU 32 operates in accordance with program instructions stored in a ROM 34, or in RAM 37, or in flash memory not shown, or on a hard drive device, not shown. A memory 36, here an erasable, or other type of programmable semiconductor memory, here a read only memory (ROM) is provided for storing a computer, here microprocessor, executable program, for operating the CPU 32. Further, the RAM 37 stores, after being read from the hard drive, not shown, a table of reference data, to be described in more detail.

Thus, the ultrasound processor 21 (FIG. 2) scan converts data associated with the radial scan pattern to generate ultrasound image data in a video format (e.g. Cartesian coordinate format). Thus, as noted briefly above, the processor 21 includes the array of transmitting/receiving elements 19, here an array of piezoelectric crystals that deliver ultrasonic energy into a patient and receive ultrasonic echoes from the patient. Electrical signals representative of the echoes produced by the transducer 12 are delivered to the beamforming network 24 where they are selectively combined to produce an indication of the echo intensity along a particular direction or beam in the patient. The data produced by the beamforming network 24 is fed to the echo processor 26 that calculates echo intensity at each position along a beam and may calculate a Doppler shift of the echoes received along a particular beam. Data from the echo processor 26 is fed to a scan converter 28 that converts the data into a form that can be readily displayed on a video monitor 22.

The data produced by the scan converter 28 is stored in an the RAM 37 where an additional processing, such as adding color in a manner to be described, is performed prior to displaying the images on the video monitor, here display 22. Controlling the operation of the above-referenced parts are one or more central processing units, here collectively indicated by the CPU 32.

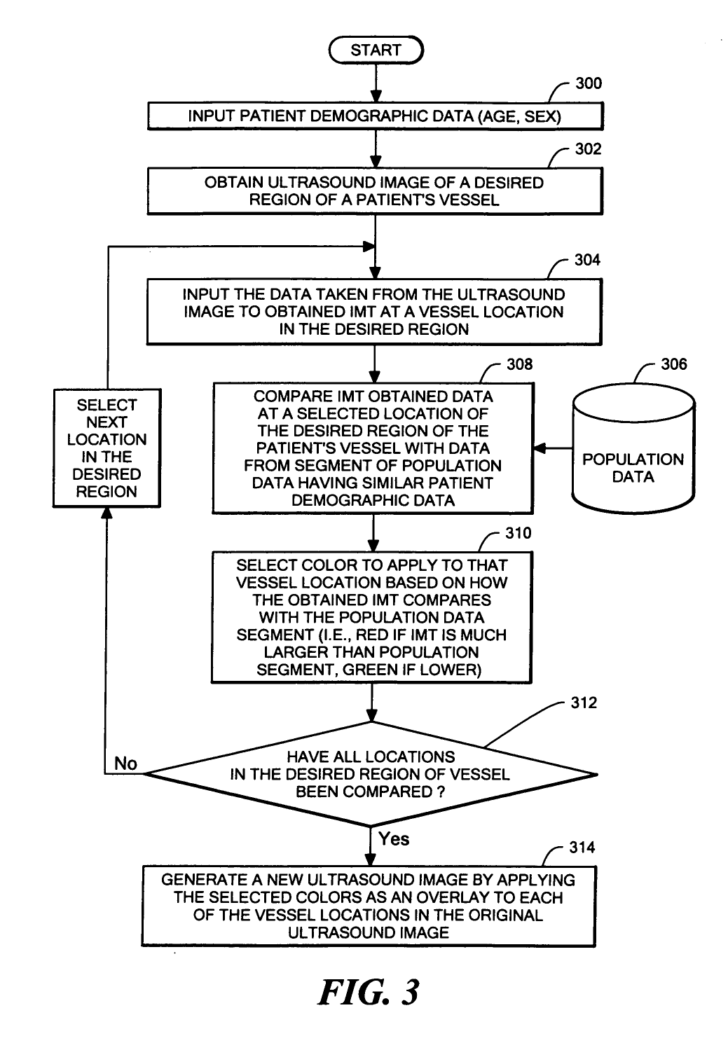

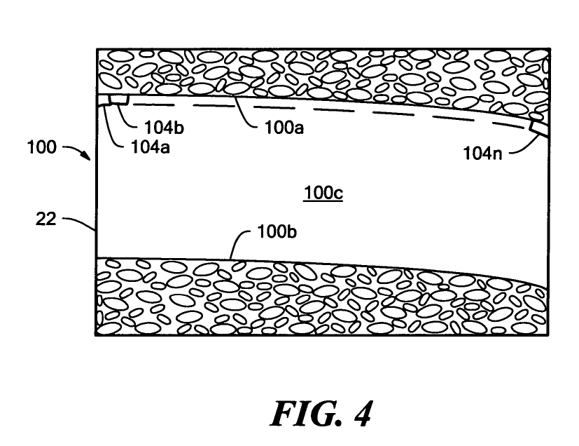

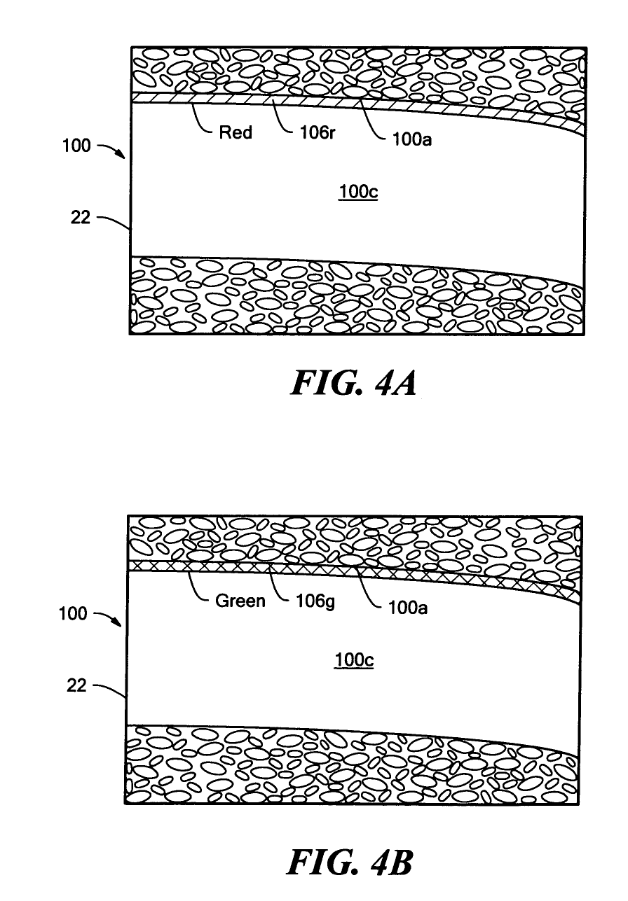

Referring now to FIG. 3, a flow diagram of the process used to display vessel wall thickness of a blood vessel, here the carotid artery, of the patient 15, FIG. 1, is shown. The process is stored as a computer program in stored in a ROM 34, or in RAM 37. The process begins by the sonographer, not shown, inputting onto one of the memories 36 or 37, FIG. 2, demographic data, i.e., age and sex, of the patient 15 (FIG. 1), Step 300. Next, an ultrasound image of a desired region of the patient's vessel is obtained, an exemplary image of such desired region of the carotid artery 100 being shown in FIG. 4, Step 302. Note the walls of the artery 100 are indicated by the numerical designation 100a, 100b, and the channel for blood flow is designated by numerical designation 100c. The ultrasound data of such desired region of the carotid artery 100 is stored in RAM 37.

Next, referring again to FIG. 3, the processor 21 uses the ultrasound data stored in RAM 37 and processes such data to generate LI and MA boundaries for the vessel wall and from the generated LI and MA boundaries determines an IMT at one selected location along the vessel wall, for example location 104a along wall 100a, FIG. 4, Step 304. FIG. 3.

Next, the process compares the IMT obtained for location 104a with data with reference data, here, in this example, data from a segment of the population statistics 306 stored, as noted above, in one of the memories 36, 37, having the same or similar demographic data, e.g., age and sex, as the patient being examined, Step 308.

The processor 21 next selects a color to apply to location 104a based on how the obtained IMT compares with the segment of the population statistics. For example, here the color red is selected if the IMT is much larger than the IMT of the population segment, the color green is selected if the IMT is lower than the IMT of the population segment, and the color yellow is selected if the IMT moderately above average for the IMT of the population segment, Step 310.

The process then determines if all locations 104b through 104n have been compared, Step 312. If not, the process selects the next location, for example, location 104b. When all locations 104a through 104n have been compared, the processor generates a new ultrasound image on display 22, FIG. 1, by applying the selected colors as an overlay to each of the vessel locations in the original ultrasound image, Step 304, as shown in FIGS. 4A and 4B, where in this example, FIG. 4A shows the color 106r of red along wall 100a and FIG. 4B shows the color 106g of green along wall 100a. It is noted that the sonographer or radiologist can easily detect by the colors displayed on display 22 that the patient having the sonogram in FIG. 4B has an abnormally high IMT whereas the patent with the having the sonogram in FIG. 4A has an acceptable IMT.

Embodiments of the present invention can also be applied to anatomical features other than the IMT. For example, the embodiments of the invention can be applied to the measurement of the length of a fetal femur, the circumference of a fetal abdomen or head, or the fetal biparietal diameter. The invention can also be applied to a measurement of the length, area, or volume of a kidney, tumor, or other anatomical feature. Other embodiments can be applied to a change or a periodic change in an area or volume, such as the cardiac left ventricular ejection fraction, or to a quantity derived from such a measurement, such as the cardiac output. In each case, a color overlay can be applied to the region of the anatomical feature, or to the numerical display of the measurement, or to an artificial rendering of the object, such as colorizing the vessel wall that is displayed with a “vessel fly-through” display. The color can be based on population statistics, the subject's age, weight, or other clinical risk factors (such as diabetes or history of smoking), one or more previous measurements, or other pre-determined criteria. Thus, the color may indicate that the size of an object is better, similar, or worse than at the time of a previous measurement. Alternatively, the color may indicate that the size of the object is normal, abnormal, healthy, diseased, or at elevated risk of future disease.

In addition to quickly conveying information to the clinician, the colorization may help to improve communication with the patient. Comments such as “the red region indicates diseased or ‘at risk’ endothelium”, or “the green on the tumor indicates that it is shrinking in size from your previous exam” may help patients to better understand the clinical implications of the pictures that they are shown. This may help improve patient compliance with proposed treatment.

A number of embodiments of the invention have been described. Nevertheless, it will be understood that various modifications may be made without departing from the spirit and scope of the invention. For example, a single color may be assigned to the entire vessel wall based on the average IMT determination and the reference IMT. Alternatively, a single color is assigned to the entire vessel wall based on the maximum determined IMT and the reference IMT. Further, the reference data may be a function of a prior IMT determination on the same patient to assess changes in the patients IMT over time. Thus, in such embodiment the reference IMT is a function of a prior IMT determination and the time duration between the current IMT determination and the prior IMT determination. Further, the measurement may be a distance, an area, a volume, a displacement, a velocity, a strain, a strain rate, or an accelerate rate. Still further, the measurement may be obtained manually (with a user interface) or automatically, or a combination thereof. Also, the database may have data manually enter by a physician of the thresholds that are of clinical interest to him, based on his own judgment and may represent healthy/deceased boundaries, for example. Accordingly, other embodiments are within the scope of the following claims.

What is claimed is:

1. A method provided for displaying a measurement associated with an anatomical feature, comprising:
determining, from ultrasound data, a measurement of such feature;
assigning a color based on a comparison between the measurement and reference data of such feature.

2. The method recited in claim 1 wherein the reference data includes population statistics.

3. The method recited in claim 1 wherein the reference data is a function of prior determined data of the feature.

4. A method provided for displaying vessel wall thickness from ultrasound imaging data, comprising:
determining from the ultrasound imaging data, Intima Media Thickness (IMT) along the vessel wall;
assigning a color is based on the Intima Media Thickness determined and a reference IMT data.

5. The method recited in claim 4 wherein a single color is assigned to the entire vessel wall based on the average intima media thickness determination.

6. The method recited in claim 4 wherein a single color is assigned to the entire vessel wall based on the maximum determined intima media thickness.

7. The method recited in claim 4 wherein the intima media thickness is determined at each of a plurality of different location along the vessel wall; and including:
assigning at each one of the different locations along the vessel wall a color based on the intima media thickness determination at each one of the different locations and the reference.

8. The method recited in claim 4 wherein the reference intima media thickness is a function of population statistics.

9. The method recited in claim 4 wherein the reference intima media thickness is a function of a prior intima media thickness determination.

10. The method recited in claim 4 wherein the reference intima media thickness is a function of a prior intima media thickness measurement and the time duration between the measured intima media thickness the prior intima media thickness measurement.

11. A method provided for displaying vessel wall thickness from ultrasound imaging data, comprising:
generating LI and MA boundaries for the vessel wall using such ultrasound imaging data;
measuring from the generated LI and MA boundaries an IMT along the vessel wall; and
assigning a color is based on the intima media thickness measurement and a reference intima media thickness.

12. The method recited in claim 11 wherein a single color is assigned to the entire vessel wall based on the average intima media thickness measurement and the reference intima media thickness.

13. The method recited in claim 11 wherein a single color is assigned to the entire vessel wall based on the maximum measured intima media thickness and the reference intima media thickness.

14. The method recited in claim 11 wherein the reference intima media thickness is a function of population statistics.

15. The method recited in claim 11 wherein the reference intima media thickness is a function of a prior intima media thickness determination.

16. The method recited in claim 11 wherein the reference intima media thickness is a function of a prior intima media thickness measurement, and the time duration between the measured intima media thickness and the prior intima media thickness measurement.

17. The method recited in claim 4 including:
generating LI and MA boundaries for the vessel wall using such ultrasound imaging data;
measuring from the generated LI and MA boundaries an intima media thickness at each of a plurality of different locations along the vessel wall; and
assigning at each one of the different locations along the vessel wall a color based on the IMT measurement at each one of the different locations.

18. The method recited in claim 17 wherein the intima media thickness reference is a function of general population statistics.

19. The method recited in claim 17 wherein the reference intima media thickness is a function of a prior intima media thickness measurement.

20. The method recited in claim 17 wherein the reference intima media thickness is a function of a prior intima media thickness measurement, and the time duration between the measured and the prior intima media thickness measurement.

21. The method recited in claim 14 wherein a single color is assigned to the entire vessel wall based on the average intima media thickness measurement.

22. The method recited in claim 14 wherein a single color is assigned to the entire vessel wall based on the maximum measured intima media thickness.

23. A method provided for displaying vessel wall thickness from ultrasound imaging data taken on a patient, comprising,
obtaining demographic information of the patient;
obtaining an ultrasound image of the vessel of the patient;
determining from the obtained image the intima media thickness of the vessel;
comparing the determined intima media thickness with data of intima media thickness for people having substantially similar demographics as the obtained patient demographic information;
selecting a color to apply to for displaying a wall of the vessel based on the comparison; and
generating an image of the patient's vessel; with the wall of the vessel image having the selected color.

24. The method recited in claim 23 wherein a single color is assigned to the entire vessel wall based on the average intima media thickness measurement.

25. The method recited in claim 23 wherein a single color is assigned to the entire vessel wall based on the maximum measured intima media thickness.

26. An ultrasound system for displaying vessel wall thickness from ultrasound imaging data taken on a patient, comprising:
a transducer, for obtaining ultrasound image data of the vessel of the patient;
a processor for:
determining from the obtained image the intima media thickness of the vessel;
comparing the determined IMT with data of intima media thickness for people having substantially similar demographics as the obtained patient demographic information;
selecting a color to apply to for displaying a wall of the vessel based on the comparison; and
generating an image of the patient's vessel; with the wall of the vessel image having the selected color.

27. The system recited in claim 26 wherein the processor assigns a single color to the entire vessel wall based on the average intima media thickness measurement.

28. The system recited in claim 26 wherein the processor assigns a single color to the entire vessel wall based on the maximum measured intima media thickness.

29. An ultrasound system for displaying a measurement associated with an anatomical feature, comprising:
a transducer for obtaining ultrasound data from such feature;
a processor for:
determining, from ultrasound data, a measurement of such feature; and
assigning a color based on a comparison between the measurement and reference data of such feature.

30. An ultrasound system for displaying a measurement associated with an anatomical feature, comprising:
a transducer for obtaining ultrasound data from such feature;
a processor for:
determining from the obtained data an image of the feature;
comparing the determined feature with data of feature for people having substantially similar demographics as the obtained patient demographic information;
selecting a color based on the comparison; and
generating an image of the feature along with the selected color.
